(19) United States
(12) Patent Application Publication
(10) Pub. No.: US 2021/0169412 A1
(43) Pub. Date: Jun. 10, 2021

(54) METHOD FOR AUTOMATICALLY RECORDING CIRCADIAN RHYTHM OF USER VIA PORTABLE DEVICE AND PORTABLE DEVICE THEREOF

(71) Applicant: National Health Research Institutes, Miaoli County (TW)

(72) Inventor: Yu-Hsuan Lin, Miaoli County (TW)

(21) Appl. No.: 16/704,997

(22) Filed: Dec. 5, 2019

(51) Int. Cl. A61B 5/00; G16H 10/65

(52) U.S. Cl. CPC A61B 5/4857; A61B 5/6898; A61B 5/7264; A61B 5/1118; A61B 5/4809; A61B 5/7225; G16H 10/65; A61B 5/7282

(57) ABSTRACT

A method for automatically recording circadian rhythm of a user via a portable device includes the steps of obtaining plural usage episodes from screen-on to successive screen-off via a screen of the portable device in an estimated period; classifying plural proactive use episodes and plural reactive use episodes from the plural usage episodes; generating active periods and inactive periods in each day via the plural proactive use episodes; and estimating daily sleep indicators from an evaluation of each of the inactive periods. Therefore, it only needs to estimate the daily sleep indicators of users by using portable devices (specifically for smartphones).

### BACKGROUND OF THE INVENTION

### 1. Field of the Invention

The invention relates to a method for automatically recording the circadian rhythm of a user via a portable device, particularly to a design for determining active periods and inactive periods in each day to estimate daily sleep indicators from each of the inactive periods in corresponding day.

### 2. Description of the Prior Art

Sleep is usually beneficial and restorative for users and has a great influence on the quality of life. The human sleep and wakefulness cycle generally conform to circadian rhythms regulated by biological clocks. Regular periods of sleep can rejuvenate and rebuild the body and mind. The body may perform various tasks during sleep, such as organizing long-term memory, integrating new information, and renewing tissue and other body structures.

For achieving sleep measurement, various wearable devices (such as several well-known wristbands on market, Fitbit® and Mi Band®) or actimetry in sleep laboratory (such as ActiWatch®) for sleep detection have been invented with the existing technology. However, these wearable devices must be synchronized with smartphones or tablets via data transmission or reception and charged regularly due to continuous recording of running power consumption and sleep interruptions in patients with sleep disorders. Additionally, the circadian rhythm of shift workers cannot be recorded by the either wearable devices or actimetry.

Furthermore, if the actimetry is used to record the circadian rhythm of a user, the standard records were carried out for two weeks at most. In this way, it is impossible to observe the stability of long-term circadian rhythms, because the long-term circadian rhythm including weekly cycle, such as the differences between week nights and weekend nights sleep. In the analysis of periodic function, the time window of raw data should cover at least three cycles that is three weeks.

Accordingly, it is necessary to provide a method which can effectively record the daily sleep indicators of the user and achieve long-term recording function, thereby solving the problems that need to be improved in the existing technology.

### SUMMARY OF THE INVENTION

In order to solve the problems described above, the object of the invention is to provide a method for automatically recording the circadian rhythm of a user via a portable device, so as to record the circadian rhythm and determine daily sleep indicators of the user.

To achieve the above object, the invention provides a method for automatically recording the circadian rhythm of a user via a portable device which includes a processor and a screen. The method is applied to the processor for executing an application, comprising the steps of: obtaining plural usage episodes from screen-on to successive screen-off via the screen in an estimated period which represents one day or more; classifying plural proactive use episodes and plural reactive use episodes from the plural usage episodes, wherein each of the plural proactive use episodes is defined as an episode that does not receive a notification in a notification threshold prior to the screen-on, and wherein each of the plural reactive use episodes is defined as an episode that receives a notification in the notification threshold prior to the screen-on, wherein the notification threshold may be within one minute; transforming the plural proactive use episodes into a periodic function; and determining at least one of the active periods and at least one of the inactive periods in each day by the periodic function;

Wherein the periodic function is transformed via a cosinor fitting.

Wherein the cosinor fitting is according to the following formula:

Cosinor fitting=a×cos(2πt/24)+b×sin(2πt/24)+c×cos(2πt/12)+d×sin(2πt/12)+e

Where a, b, c, d and e represent constants, t represents time, the value of denominators represents the time in hours which is adjustable; estimating daily sleep indicators from each of the inactive periods, wherein the plural reactive use episodes are excluded when the daily sleep indicators is estimated in each of the inactive periods.

Wherein the daily sleep indicators are estimated through one of longest gaps between two of the plural proactive use episodes in the inactive periods. Wherein a wake time in the daily sleep indicator is determined by at least one of the wake time determination factors, and when the proactive use episode is not conformed to the wake time determination factor, the wake time will be set on another proactive use episode which conforms to the wake time determination factor.

Wherein the wake time determination factor includes the duration of the proactive use episode exceeding a first awakening threshold, the using parameter exceeding a second awakening threshold, a duration weighted value being below a third awakening threshold, or any of their combinations; wherein the using parameter is an added value of the duration of the proactive use episode and a weighted value of application usage times, and the duration weighted value is determined by the duration between the proactive use episode and the start point of the active period.

When the duration between the proactive use episode and the start point of the active period is shorter, the duration weighted value is smaller. When the duration between the proactive use episode and the start point of the active period is longer, the duration weighted value is greater.

Wherein the method further comprises: receiving plural self-reports by an input module; generating a report deviation according to a differential value between time average of plural daily sleep indicators and time average of the corresponding plural self-reports; and adjusting the daily sleep indicators by the report deviation.

Wherein the processor adjusts a sleep onset time or a wake time in the daily sleep indicators ahead or delayed according to the report deviation.

Wherein the processor executes an application to provide an input surface at a daily reporting time to allow the user to input the plural self-reports to the input module.

Wherein the input module is located in the portable device and connected with the processor to receive the plural self-reports.

Wherein the method further comprises: receiving plural sleep measurements by a sleep measuring device; generating a measurement deviation according to a differential value between time average of plural daily sleep indicators and time average of the corresponding plural sleep measurements; an adjusting the daily sleep indicators by the measurement deviation.

Wherein the processor adjusts a sleep onset time or a wake time in the daily sleep indicators ahead or delayed according to the measurement deviation.

In order to solve the problems described above, another object of the invention is to provide a portable device for automatically recording the circadian rhythm of a user, including a screen configured to display an image and determine an action of screen-on or screen-off, and a processor located in the portable device for applying to execute the aforesaid method.

Wherein the portable device further comprises: an input module connected to the processor for receiving a self-report input from the user; and a recording module connected to the processor for generating a recording chart based on the daily sleep indicators.

Wherein the portable device connects to a sleep measuring device for capturing a sleep measurement in each day, and the processor in the portable device generates a measurement deviation according to a differential value between the daily sleep indicators and each of plural sleep measurements in corresponding day, then the processor adjusts the daily sleep indicators by the measurement deviation.

### BRIEF DESCRIPTION OF THE DRAWINGS

The techniques of the present invention can be more easily understood from the detailed description given below, and the accompanying drawings are provided for better illustration, and thus the description and the accompanying drawings are not restrictive to the present invention.

FIG. 1 is a schematic diagram for obtaining plural usage episodes according to the present invention.

FIG. 2 is a schematic diagram for classifying plural proactive use episodes and plural reactive use episodes according to the present invention.

FIG. 3 is a fitting chart of a cosinor fitting according to the present invention.

FIG. 4 is a schematic diagram of daily sleep indicators according to the present invention.

FIG. 5 is a flowchart for obtaining the daily sleep indicators of a user according to the present invention.

FIG. 6 is a schematic diagram for adjusting the daily sleep indicators by a self-report according to the present invention.

FIG. 7 is a schematic diagram for showing a recording chart on a screen of a portable device according to the present invention.

FIG. 8A is a block diagram of one embodiment of the portable device according to the present invention.

FIG. 8B is a block diagram of another embodiment of the portable device according to the present invention.

FIG. 9 is a block diagram of the portable device connected to a sleep measuring device according to the present invention.

### DETAILED DESCRIPTION OF THE PREFERRED EMBODIMENTS

The aspects of the invention are now described in the following preferred embodiments; however, the invention is not limited thereto.

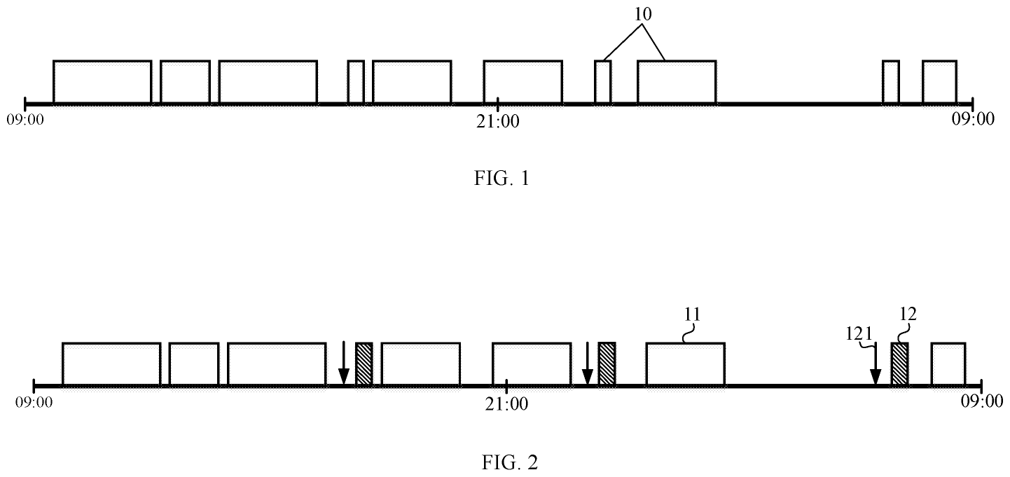

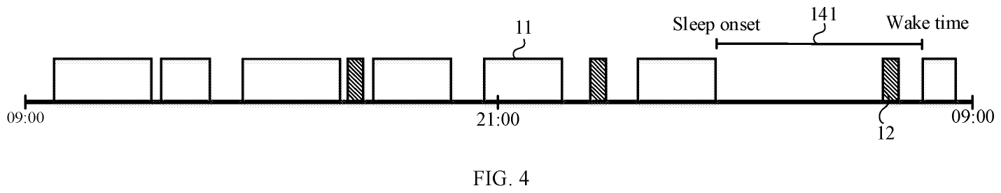

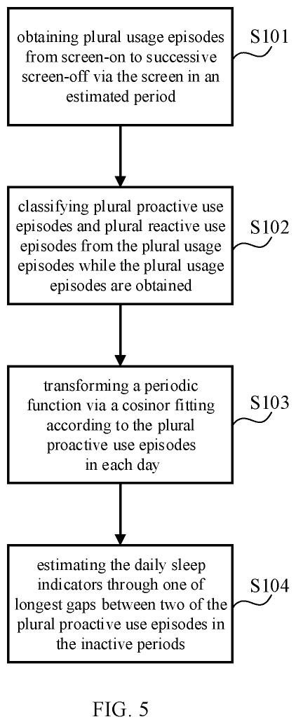

Referring to FIG. 1 to FIG. 5, which illustrate respectively schematic diagrams for obtaining plural usage episodes, classifying plural proactive use episodes and plural reactive use episodes, and daily sleep indicators, a fitting chart of a cosinor fitting, and a flowchart for obtaining the daily sleep indicators of a user of the invention. As shown in the FIG. 1 to FIG. 5, since the use of portable devices (e.g. smartphones) is prevalent worldwide, the daily sleep indicators 141 can be estimated by the portable device at present. Therefore, the invention mainly provides a method for automatically recording the circadian rhythm of a user to estimate the daily sleep indicators 141 of the user via the portable device which includes a processor and a screen. The method is applied to the processor that executes an application, wherein the method can be achieved by the following steps of:

S101: obtaining plural usage episodes 10 from screen-on to successive screen-off via the screen in an estimated period (one day or more); because people have to turn the screen on at the beginning of using the portable device and turn the screen off at the end of using the portable device, the plural usage episodes 10 can be identified by the successive action from screen-on to screen-off.

S102: classifying plural proactive use episodes 11 and plural reactive use episodes 12 from the plural usage episodes 10 while the plural usage episodes 10 are obtained; wherein a proactive use refers to the user who actively uses the portable device, while a reactive use refers to the user who passively uses the portable device (e.g. receive a message notification or a clock alarm). In the invention, each of the plural proactive use episodes 11 is further defined as an episode that does not receive a notification 121 in a notification threshold (e.g. within about one minute) prior to the screen-on, while each of the plural reactive use episodes 12 is defined as an episode that receives a notification 121 in the notification threshold prior to the screen-on to distinguish between two types of the proactive use and the reactive use.

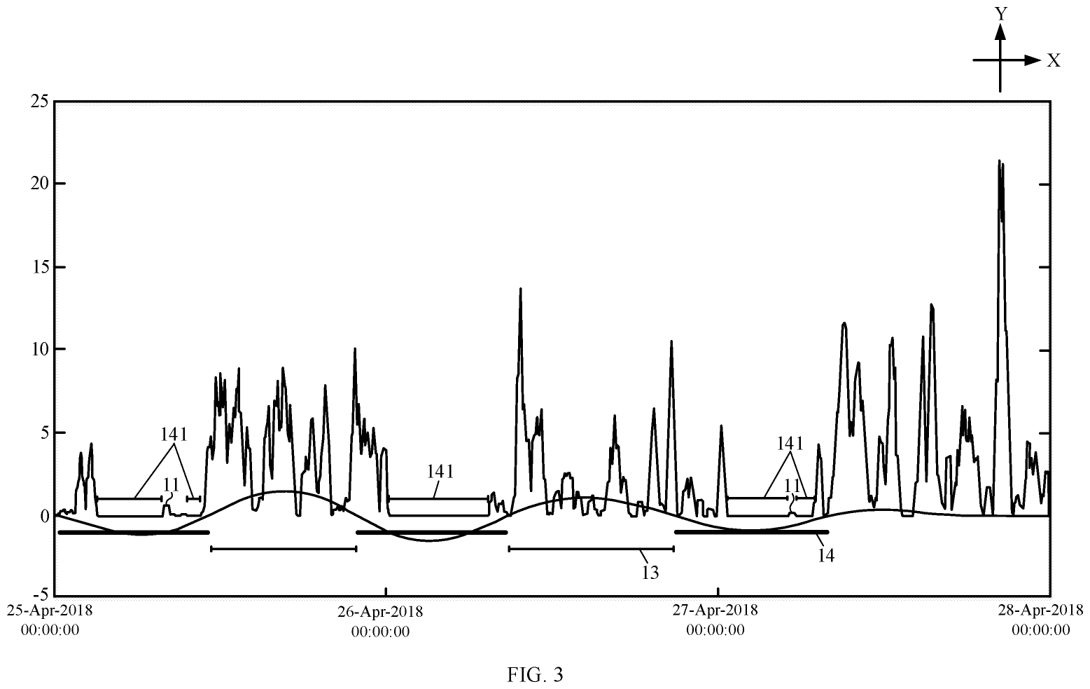

S103: transforming the plural proactive use episodes 11 into a periodic function 20. In one embodiment, the periodic function may be transformed via a cosinor fitting, wherein the cosinor fitting is used for illustrating the periodic function 20 through fitting the plural proactive use episodes 11 in each day to correspond to the daily circadian rhythm of the user to determine at least one of active periods 13 (such as crests in FIG. 3) and at least one of inactive periods 14 (such as troughs in FIG. 3). In one embodiment, the active periods 13 and the inactive periods 14 may be recognized via dividing the periodic function 20 horizontally (X-axis) which starts at the 0 value of the crests which above the division line are treated as the active periods 13, and the troughs which beneath the division line are treated as the inactive periods 14.

In one embodiment, the cosinor fitting may be determined according the following formula:

Cosinor fitting=a×cos(2πt/24)+b×sin(2πt/24)+c×cos(2πt/12)+d×sin(2πt/12)+e

Where a, b, c, d and e represent constants (each can be any number) which are used to fit the periodic function 20, t represents time, and the value of denominators (e.g. 24 and 12 as above) represents the time in hours which can be adjusted depending on the usage status of the portable device to present the periodic function 20.

S104: estimating the daily sleep indicators 141 through one of longest gaps between two of the plural proactive use episodes 11 in the inactive periods 14, wherein the plural reactive use episodes 12 are excluded when the daily sleep indicators 141 is estimated.

During the sleep, the user may wake up to use the portable device in a fragmented manner, so to avoid such fragmented use being treated as wake time, the wake time in the daily sleep indicator 141 is determined by at least one of wake time determination factors, when the proactive use episode 11 is not conformed to the wake time determination factor, the proactive use episode 11 may be treated as the fragmented use, and the wake time will be set on another proactive use episode 11 which conforms to the wake time determination factor.

The wake time determination factor includes the duration of the proactive use episode exceeding a first awakening threshold (e.g. 20 minutes), the using parameter (the Y-axis as shown in FIG. 3) exceeding a second awakening threshold (e.g. the value of 3), a duration weighted value being below a third awakening threshold (e.g. lower than 15%) or any of their combinations, wherein the using parameter is an added value of the duration of the proactive use episode and a weighted value of application usage times, the duration weighted value is determined by the duration between the proactive use episode 11 and the start point of the active periods 13.

For instance, when one of the proactive use episode 11 is obtained at 5:00 a.m. (which the start point of the active periods 13 right at 6:00 a.m.), the duration weighted value may be 20%, and if another proactive use episode 11 is obtained at 5:30 a.m., the duration weighted value may be 10% and so on. Therefore, when the duration between the proactive use episode 11 and the start point of the active periods 13 is shorter, the duration weighted value may correspond to be smaller, when the duration between the proactive use episode and the start point of the active periods 13 is longer, the duration weighted value may correspond to be greater.

When the daily sleep time is estimated basing on each of the daily sleep indicators 141, the plural proactive use episodes 11 which are not conformed to the wake time determination factor in the daily sleep indicators 141 may be deducted. For example, if one day of the daily sleep indicator 141 is indicated on 10:00 p.m. to 6:00 a.m. and one of the proactive use episode 11 which is not conformed to the wake time determination factor is indicated on 5:00 a.m. to 5:10 a.m., the daily sleep indicator 141 of the day may deduct the proactive use episode 11 to be separated into two sections of 10:00 p.m. to 5:00 a.m. and 5:10 a.m. to 6:00 a.m. Therefore, when the daily sleep time of the day is estimated based on the daily sleep indicator 141 of the day, the duration of the proactive use episode 11 (10 minutes) may be deducted so that the daily sleep time of the day may be 7 hours and 50 minutes.

Through the above method, the daily sleep indicators 141 and the daily sleep time of the user can be estimated via the portable device.

When the processor obtains the daily sleep indicators 141 of at least two weeks, the daily sleep indicators 141 of a week will be compared with another week through the standard deviation, empirical mode decomposition, social jetlag, or any of their combinations to generate long-term analysis data.

Accordingly, the long-term circadian rhythm of the user can be observed or delineated by the long-term analysis data, and the daily sleep indicators 141 can be estimated through the cosnior fitting whether the circadian rhythm of the user is regular or not.

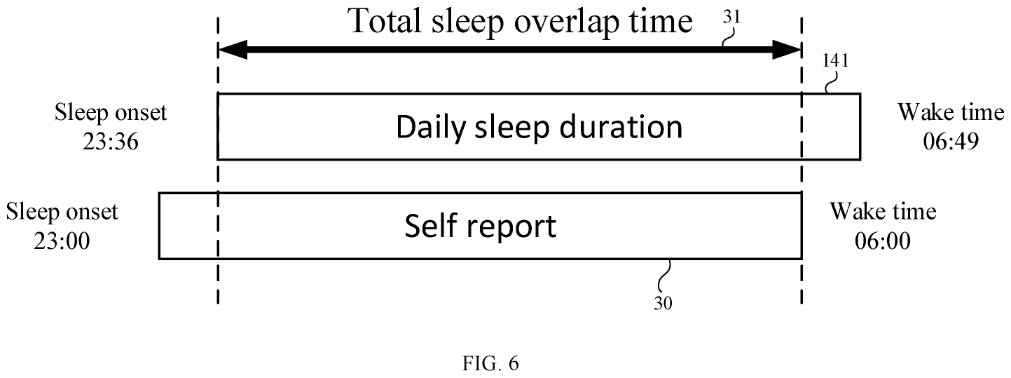

Referring to FIG. 6, which illustrates a schematic diagram for adjusting the daily sleep indicators by a self-report of the invention. As shown in the FIG. 6, under common circumstances, the user may not fall asleep immediately after turning the screen off or use the portable device immediately after waking up. Therefore, in order to adjust the daily sleep indicators 141 of the user, the invention further provides an input module which is located in the portable device and connected with the processor to receive data from the user. After the user wakes up, the processor executes an application to provide an input surface at a daily reporting time (e.g. 10 a.m.) to allow the user to input the self-report 30 (which is actual sleep indicators) to the input module. When plural self-reports 30 are input, the processor compares the daily sleep indicators 141 with each of the plural self-reports 30 in corresponding day, and generates a report deviation according to a differential value between the daily sleep indicators 141 and each of the plural self-reports 30 in corresponding day. Finally, the processor adjusts the daily sleep indicators 141 by the report deviation.

Assuming that a time average of the daily sleep indicators 141 in weekly estimated from sleep onset time is 242.9 seconds delayed from the corresponding plural self-reports 30, the processor adjusts the daily sleep indicators 141 by generating the report deviation to set the sleep onset time to 242.9 seconds ahead. Under another assumption, if a time average weekly wake time during the time average of the daily sleep indicators in weekly 141 is 623.7 seconds earlier from the corresponding wake time of the plural self-reports 30, the processor adjusts the daily sleep indicators 141 by generating the report deviation to set the wake time to a 623.7 second delay.

According to the above adjustment, an overlap ratio of the daily sleep indicators 141 and each of the plural self-reports 30 in corresponding day can reach more than 90% consistency, which is verified by the following formula:

Overlap ratio=total sleep overlap time÷((the self-report of the day+the daily sleep indicator of the day)÷2)×100%

Where the total sleep overlap time 31 is an overlap time between the daily sleep indicator 141 of the day and the self-report 30 of the day.

Thus, the invention ensures that the daily sleep indicators 141 is closer to the actual sleep time of the user through the above adjustment.

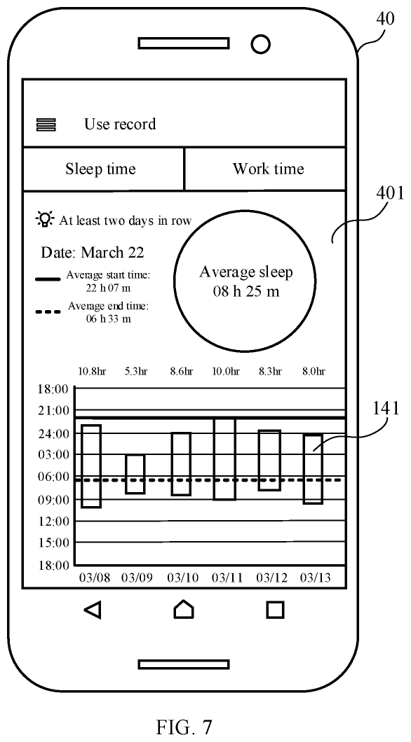

Referring to FIG. 7, which illustrates a schematic diagram for showing a recording chart on a screen of the portable device of the invention. As shown in the FIG. 7, when the daily sleep indicators 141 of two days or more are obtained via the portable device 40, the processor outputs the daily sleep indicators 141 to a recording module to generate a recording chart 401 of each day based on the daily sleep indicators 141, which can be provided to users or Health care professionals (HCPs) to observe and record their daily circadian rhythms.

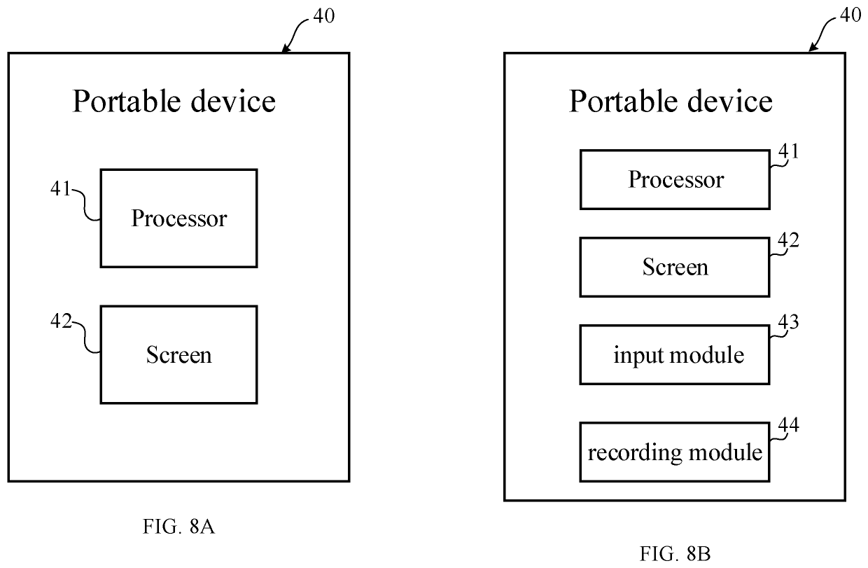

Referring to FIGS. 8A and 8B, block diagrams of the portable device of the invention are shown, respectively. As shown in the FIGS. 8A and 8B, to achieve the method for estimating the daily sleep indicators 141 of the user as described above, the invention provides one embodiment of the portable device 40 which may be, for example, a smartphone, a tablet, a wearable electronic device, and may be configured with or without communication function. In this embodiment, the portable device 40 is a smartphone with communication function between the processor 41 and the screen 42, the processor 41 is located in the portable device 40 for applying the method and executing the application, and the screen 42 is configured to display an image and determine the action of screen-on or screen-off (as shown in FIG. 8A).

The invention further provides another embodiment of the portable device 40 which includes an input module 43 connected to the processor 41 for receiving data (specifically for the self-report) input from the user, and a recording module 44 connected to the processor 41 for generating the recording chart based on the daily sleep indicators 141 (as shown in FIG. 8B).

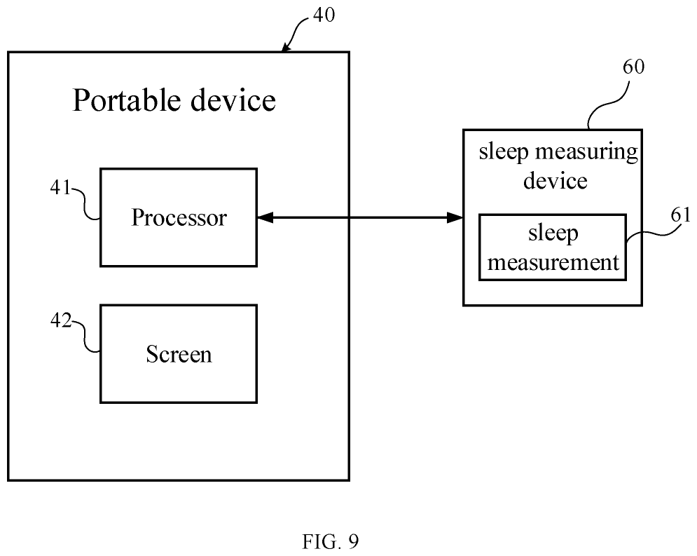

Referring to FIG. 9, which illustrates a block diagram of the portable device connected to a sleep measuring device of the invention. As shown in the FIG. 9, the portable device 40 is further connected to the sleep measuring device 60 (such as actimetry or wearable device) to capture a sleep measurement 61 in each day, then the processor adjusts the daily sleep indicators 141 by a measurement deviation in a way of a self-report adjustment, wherein the measurement deviation is a differential value between time average of the plural daily sleep indicators 141 and time average of corresponding plural sleep measurements 61, thereby adjusting the daily sleep indicators 141 of the user in various ways.

Accordingly, the daily sleep indicators 141 in the invention may at least include the sleep onset time, the wake time, the proactive use episode(s) 11 which is/are determined as the fragmented use.

In the above description, for explanation, numerous specific details are outlined to provide a thorough understanding of the embodiments. However, it is apparent to those skilled in the art that one or more other embodiments may be practiced without some of these specific details. It should be appreciated that references throughout this specification to “one embodiment”, “an embodiment” and embodiments with an indication of ordinal numbers and so forth mean that particular features, structures, or characteristics may be included in the implementation of the invention. It should be further appreciated that in the description, various features are sometimes grouped in a single embodiment, drawing, or description thereof to streamline the invention and facilitate understanding of various inventive aspects.

While the invention has been described in connection with what is considered to be the exemplary embodiments, it is understood that this invention is not limited to the disclosed embodiments, but is intended to cover various arrangements including the spirit and scope of the broadest interpretation so as to encompass all such modifications and equivalent arrangements.

What is claimed is:

1. A method for automatically recording circadian rhythm of a user via a portable device which includes a processor and a screen, the method being applied to the processor for executing an application and comprising:
obtaining plural usage episodes from screen-on to successive screen-off via the screen in an estimated period, wherein the estimating period represents one or more days;
classifying plural proactive use episodes and plural reactive use episodes from the plural usage episodes, wherein each of the plural proactive use episodes is defined as an episode with no notification received in a notification threshold prior to the screen-on, and wherein each of the plural reactive use episodes is defined as an episode with a notification received in the notification threshold prior to the screen-on;
determining at least one of active periods and at least one of inactive periods in each day via the plural proactive use episodes; and
estimating daily sleep indicators from each of the inactive periods.

2. The method of claim 1, wherein the plural reactive use episodes are excluded when the daily sleep indicators are estimated in each of the inactive periods.

3. The method of claim 1, wherein the processor transforms the plural proactive use episodes into a periodic function, and determines each of the active periods and each of the inactive periods by the periodic function.

4. The method of claim 3, wherein the periodic function is transformed via a cosinor fitting.

5. The method of claim 4, wherein the cosinor fitting is determined according to the following formula:
Cosinor fitting=a×cos(2πt/24)+b×sin(2πt/24)+c×cos(2πt/12)+d×sin(2πt/12)+e
where a, b, c, d and e represent constants, t represents time, and the value of denominator represents the time in hours which is adjustable.

6. The method of claim 1, wherein the daily sleep indicators are estimated through one of longest gaps between two of the plural proactive use episodes in the inactive periods.

7. The method of claim 6, wherein a wake time in the daily sleep indicator is determined by at least one of wake time determination factors, and when the proactive use episode is not conformed to the wake time determination factor, the wake time is set on another proactive use episode which conforms to the wake time determination factor.

8. The method of claim 7, wherein the wake time determination factor includes a duration of the proactive use episode exceeding a first awakening threshold, a using parameter exceeding a second awakening threshold, a duration weighted value being below a third awakening threshold, or any of their combinations, wherein the using parameter is an added value of the duration of the proactive use episode and a weighted value of application usage times, and the duration weighted value is determined by a duration between the proactive use episode and a start point of the active period.

9. The method of claim 8, when the duration between the proactive use episode and the start point of the active period is shorter, the duration weighted value is smaller, and when the duration between the proactive use episode and the start point of the active period is longer, the duration weighted value is greater.

10. The method of claim 1, wherein the method further comprises:
receiving plural self-reports by an input module;
generating a report deviation according to a differential value between a time average of plural daily sleep indicators and a time average of corresponding plural self-reports; and
adjusting the daily sleep indicators by the report deviation.

11. The method of claim 10, wherein the processor adjusts a sleep onset time or a wake time in the daily sleep indicators ahead or delayed according to the report deviation.

12. The method of claim 10, wherein the processor executes an application to provide an input surface at a daily reporting time to allow the user to input the plural self-reports to the input module.

13. The method of claim 12, wherein the input module is located in the portable device and connected with the processor to receive the plural self-reports.

14. The method of claim 1, wherein the method further comprises:
receiving plural sleep measurements by a sleep measuring device;
generating a measurement deviation according to a differential value between a time average of plural daily sleep indicators and a time average of corresponding plural sleep measurements; and
adjusting the daily sleep indicators by the measurement deviation.

15. The method of claim 14, wherein the processor adjusts a sleep onset time or a wake time in the daily sleep indicators ahead or delayed according to the measurement deviation.

16. A portable device for automatically recording circadian rhythm of a user, comprising:
a screen configured to display an image and determine an action of screen-on or screen-off;
a processor located in the portable device for applying to execute the method as claimed in claim 1.

17. The portable device of claim 16, wherein the portable device further comprises:
an input module connected to the processor for receiving a self-report input from the user; and
a recording module connected to the processor for generating a recording chart based on the daily sleep indicators.

18. The portable device of claim 16, wherein the portable device connects to a sleep measuring device for capturing a sleep measurement in each day, and the processor in the portable device generates a measurement deviation according to a differential value between the daily sleep indicators and each of plural sleep measurements in a corresponding day, then the processor adjusts the daily sleep indicators by the measurement deviation.
